(19) United States
(12) Patent Application Publication
(10) Pub. No.: US 2016/0354009 A1
(43) Pub. Date: Dec. 8, 2016

(54) Minimally Invasive Patient Reference Systems and Methods for Navigation-Assisted Surgery

(71) Applicant: General Electric Company, Schenectady, NY (US)

(72) Inventor: Tobias Schroeder, Melrose, MA (US)

(21) Appl. No.: 14/730,892

(22) Filed: Jun. 4, 2015

(51) Int. Cl. A61B 5/06; A61B 5/00; A61B 17/86

(52) U.S. Cl. CPC A61B 5/062; A61B 17/86; A61B 5/6833; A61B 2019/524

(57) ABSTRACT

Minimally invasive systems and methods for navigation-assisted surgery are described. The system comprises a patient reference device and a transmitter. The patient reference device comprises a microsensor disposed at a distal end. The distal end is configured to pierce a patient's skin and to be anchored in a patient's anatomy. The microsensor is at least partially embedded in the patient's anatomy and is anchored proximate to a surgical field of interest. The transmitter is configured to be detachably attached to the patient's skin proximate to the surgical field of interest. The system establishes a global navigation reference frame by transmitting signals from the transmitter and receiving the signals with the microsensor to establish a position and an orientation of the microsensor to register a position and an orientation of the patient's anatomy within the global navigation reference frame. Other embodiments are described.

### FIELD

This application generally relates to navigation-assisted surgery systems (or surgical navigation systems). In particular, this application relates to minimally invasive patient reference systems and methods for navigation-assisted surgery, including a patient reference device with one or more sensors placed inside the human body.

### BACKGROUND

Navigation-assisted surgery often involves image-guided surgery with navigated instruments. Image-guided surgery systems track the precise location of surgical instruments in relation to multidimensional images of a patient's anatomy. Additionally, image-guided surgery systems use visualization tools to provide the surgeon with co-registered views of these surgical instruments with the patient's anatomy. The multidimensional images of a patient's anatomy may include computed tomography (CT) imaging data, magnetic resonance (MR) imaging data, positron emission tomography (PET) imaging data, ultrasound imaging data, X-ray imaging data, or any other suitable imaging data, as well as any combinations thereof.

While navigation-assisted surgery can assist the surgeon to place and manipulate surgical instruments that are internal to the body and difficult to view during the procedure, the registration of the multidimensional images, the surgical instruments, and patient anatomy can be a challenging problem. Conventional registration of the multidimensional images, the surgical instruments, and the patient anatomy to a global navigation reference frame is facilitated by attachment of an external patient reference device to the patient. Typically, the external patient reference device comprises a relatively large sensor or reference array device affixed by a sturdy attachment to a portion of the patient's bony anatomy.

For example, to secure conventional optical or electromagnetic external patient reference devices in spine surgery, the surgeon creates a surgical incision in the patient's back and removes tissue surrounding a vertebral body. The surgeon then attaches the external patient reference device to the vertebral body with a spine clamp or similar device. The external patient reference device remains outside of the body and is connected to the spine clamp or similar device by a relatively long rod that traverses the surgical incision.

### SUMMARY

This application describes minimally invasive systems and methods for navigation-assisted surgery. The system comprises a patient reference device and a transmitter. The patient reference device comprises a microsensor disposed at a distal end. The distal end is configured to pierce a patient's skin and to be anchored in a patient's anatomy. The microsensor is at least partially embedded in the patient's anatomy and is anchored proximate to a surgical field of interest. The transmitter is configured to be detachably attached to the patient's skin proximate to the surgical field of interest. The system establishes a global navigation reference frame by transmitting signals from the transmitter and receiving the signals with the microsensor to establish a position and an orientation of the microsensor to register a position and an orientation of the patient's anatomy within the global navigation reference frame.

### BRIEF DESCRIPTION OF THE DRAWINGS

The following description can be better understood in light of the Figures, in which:

FIG. 1 is a schematic diagram of embodiments of a minimally-invasive patient reference system for navigation-assisted surgery;

FIG. 2 is a schematic view of embodiments of a minimally invasive patient reference device;

FIG. 3 is a schematic view of embodiments of a distal tip of a minimally invasive patient reference device;

FIG. 4 is a schematic view of embodiments of a transmitter module;

FIG. 5 is a flow diagram of embodiments of a method for navigation-assisted surgery employing a minimally invasive patient reference device; and

FIG. 6 is a schematic diagram of embodiments of a minimally-invasive patient reference system for navigation-assisted surgery.

The Figures illustrate specific aspects of minimally invasive systems and methods for navigation-assisted surgery. Together with the following description, the Figures demonstrate and explain the principles of the structures, methods, and principles described herein. In the drawings, the thickness and size of components may be exaggerated or otherwise modified for clarity. The same reference numerals in different drawings represent the same element, and thus their descriptions will not be repeated. Furthermore, well-known structures, materials, or operations are not shown or described in detail to avoid obscuring aspects of the described devices.

As the terms on, attached to, or coupled to are used herein, one object (e.g., a material, a layer, a substrate, etc.) can be on, attached to, or coupled to another object regardless of whether the one object is directly on, attached, or coupled to the other object or there are one or more intervening objects between the one object and the other object. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

### DETAILED DESCRIPTION

The following description supplies specific details in order to provide a thorough understanding. Nevertheless, the skilled artisan will understand that the described minimally invasive systems and methods for navigation-assisted surgery can be implemented and used without employing these specific details. Indeed, the described minimally invasive systems and methods for navigation-assisted surgery can be placed into practice by modifying the described systems and methods and can be used in conjunction with any other apparatus and techniques conventionally used in the industry. For example, while the description below focuses on tracking instruments used in surgical navigation systems, the methods and systems for tracking instruments can be used in other systems in the fields of biomechanics, ergonomics, flight simulation and flight training, virtual reality applications, etc.

As the terms on, attached to, or coupled to are used herein, one object (e.g., a material, a layer, a substrate, etc.) can be on, attached to, or coupled to another object regardless of whether the one object is directly on, attached, or coupled to the other object or there are one or more intervening objects between the one object and the other object. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

In various embodiments, a system and method for displaying the real-time state of an imaged surgical area of interest and accurately tracking and displaying surgical instruments during a surgical procedure are disclosed. The system and method combines navigation-assisted surgery (e.g. surgical navigation) with a minimally invasive patient reference device. The minimally invasive patient reference device allows for the registration of one or more of multidimensional images of the area of surgical interest, navigated surgical tools, and a patient's anatomy in the surgical area of interest to a global reference frame. The minimally invasive patient reference device can facilitate the registration of the different navigated surgery components by being anchored to a patient's anatomy in the surgical field of interest.

The systems and methods refer to selected surgical procedures using bony anatomy as an anchor point for a distal end of the minimally invasive patient reference device. However, it should be appreciated that the systems and methods need not be limited to any surgical procedures or to only surgical areas of interest with proximate bony anatomy. The systems and methods described may be used in any surgical procedure where registration of one or more of multidimensional images of the area of surgical interest, navigated surgical tools, and a patient's anatomy in the surgical area of interest such as bony anatomy to a global reference frame is required. Likewise, the systems and methods may be used in surgical areas of interest where no proximate bony anatomy is present by anchoring the distal end of the minimally invasive patient reference device to patient's anatomy in tissue proximate to the surgical area of interest. Similarly, the systems and methods described may be used in surgical areas of interest comprising and/or surrounded by soft tissue.

In navigation-assisted surgery, the surgeon accesses the surgical area of interest by one or more percutaneous incisions. In some cases, the nature of the navigated surgery may require one or more larger incisions in the body of a patient. The surgeon inserts navigated surgical tools through these incisions and guides them to the surgical area of interest. The surgeon uses the navigation-assisted surgery technology to help guide the navigated surgical tools to the area of surgical interest and to further manipulate the navigated surgical tools to accomplish the surgical procedure. The navigation-assisted surgery technology measures the real-time location and/or orientation of the navigated surgical tool(s) and virtually superimposes the real-time location and/or orientation of the navigated surgical tool(s) on an image of the surgical area of interest. The image may be a pre-acquired image, or an image obtained in near real-time or real-time using known imaging technologies such as computed tomography (CT), magnetic resonance imaging (MRI), fluoroscopic images, positron emission tomography (PET), ultrasound, X-ray, or any other suitable imaging technology, as well as any combinations thereof. In some embodiments, the image can comprise a combination or composite of one or more pre-acquired images and one or more real-time or near real-time images.

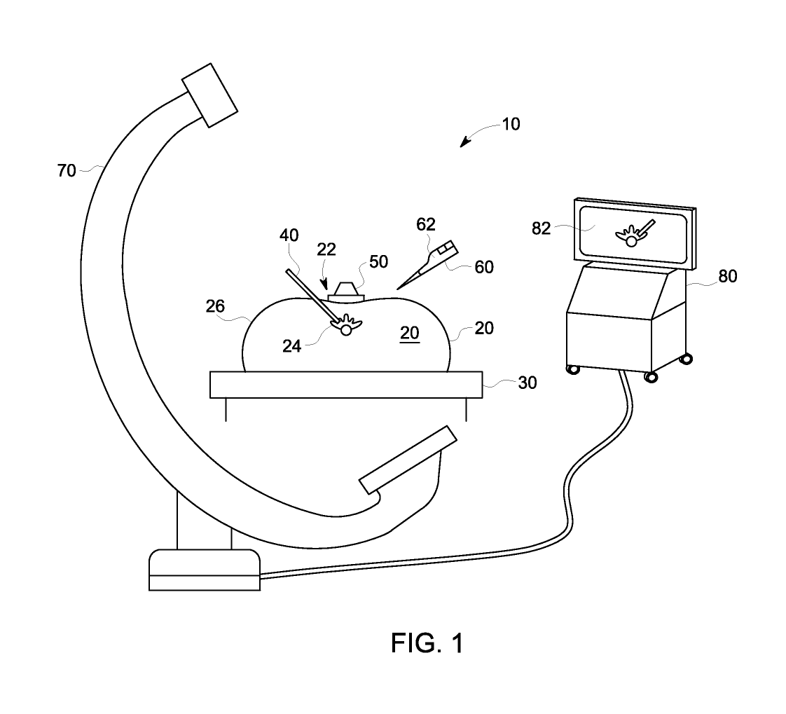

Referring to FIG. 1, some embodiments of a minimally invasive patient reference system for navigation-assisted surgery 10 are illustrated. The system 10 can include a patient 20 positioned on an operating table 30. The patient 20 is positioned such that a surgeon can access a surgical field of interest 22 and a patient's anatomy 24 proximate to the surgical field of interest 22. The system 10 can also include a minimally invasive patient reference device 40. The minimally invasive patient reference device 40 can comprise an elongated bone pin or stylet with a microsensor disposed at a distal end. The surgeon places the minimally invasive patient reference device 40 by piercing a surface of the patient's skin 26 with the distal end of the minimally invasive patient reference device 40, traversing intervening tissue, and anchoring the distal end to patient's anatomy 24.

The system 10 can also include a transmitter 50 configured to be attached to the surface of the patient's skin 26 at an area proximate to the surgical field of interest 22 and proximate to the position of the distal end of the minimally invasive patient reference device 40. The system 10 can further comprise a navigated surgical tool 60 with a receiver 62. The system 10 can further comprise an imaging assembly 70 configured to image the surgical field of interest 22. The system 10 can also comprise a workstation 80 configured with a display 82 for displaying one or more images of the surgical field of interest 22, the bony anatomy 24, the minimally invasive patient reference device 40, and the navigated surgical tool 60.

Referring now to FIG. 2, some embodiments of a minimally invasive patient reference device 40 are illustrated. The minimally invasive patient reference device 40 can comprise an elongate body 42 with a distal end 43 and a proximal end 44 (as viewed from the point of view of the surgeon placing the minimally invasive patient reference device 40). In some embodiments, the elongate body 42 can be configured in the shape of a bone pin. In other embodiments, the elongate body 42 can be configured as a bone stylet. In yet other embodiments, the elongate body 42 can be configured as a bone screw. In some embodiments, a length of the elongate body 42 can be configured to minimize a portion of the elongate body 42 remaining outside of the surface of the patient's skin 26. In other embodiments, the length of the elongate body 42 can be configured to minimize the risk of accidental impact by medical personnel during a navigated surgical procedure. In yet other embodiments, the length of the elongate body 42 can be configured to minimize the risk of accidental dislocation and/or detachment of the minimally invasive patient reference device 40. In some embodiments, the length of the elongate body 42 can be configured such that the entire length of the elongate body 42 is embedded in the patient's tissue.

In some embodiments, the distal end 43 can comprise a point 45. The point 45 can be configured to pierce the surface of the patient's skin 26 at an area proximate to the surgical field of interest 22. The point 45 can also be configured to pierce the surface of the patient's skin 26 and to make an incision in the patient's tissue while traversing the patient's tissue. In some embodiments, the minimally invasive patient reference device 40 can be configured to be anchored in a patient's anatomy 24 without the need to remove the patient's tissue. In other embodiments, the minimally invasive patient reference device 40 can be configured to be anchored in a patient's anatomy 24 by removing only a de minimus amount of the patient's tissue.

The minimally invasive patient reference device 40 further comprises a microsensor 46 disposed at the distal end 43. In some embodiments, the microsensor 46 can be configured to receive signals from the transmitter 50. In some embodiments, the microsensor 46 can comprise an electromagnetic sensor or an electromagnetic receiver such as an electromagnetic receiver configured to receive electromagnetic signals from the transmitter 50. In some embodiments, the microsensor 46 can be configured to provide sufficient number of sensitivity axes to calculate six degrees of freedom (three translational parameters and three rotation parameters). In other embodiments, the microsensor 46 can comprise magnetic pickup coils, magnetoresistive sensors, fluxgate magnetometers, six-degree of freedom induction microsensors, or combinations thereof.

In some configurations, the microsensor 46 can comprise a sensor electrical lead 48 configured to electrically connect the microsensor 46 to the workstation 80. The elongate body 42 can be configured with a hollow portion to accommodate the sensor electrical lead 48. The sensor electrical lead 48 can extend from the proximal end 44 to the workstation 80 so that it electrically connects the microsensor 46 to a wireless module configured to wirelessly communicate with the workstation 80.

In some embodiments, the proximal end 44 can be configured to facilitate handling and manipulation by the surgeon. The surface of the proximal end 44 can be textured or otherwise configured to allow for adequate grip and non-slippage by the surgeon during placement and/or anchoring. The proximal end 44 can also be configured to detachably receive other surgical tools to facilitate piercing of the surface of the patient's skin 26. For example, the proximal end 44 can be configured to detachably receive a chuck of a surgical drill or an adaptor connected to a chuck of a surgical drill. The proximal end 44 can be connected to the surgical drill and the surgical drill activated to facilitate piercing of the surface of the patient's skin 26, traversing of tissue, and/or anchoring of the distal end 43. The proximal end 44 can also be configured to detachably receive a handle, extension, adapter, or similar device to assist the surgeon in placement of the minimally invasive patient reference device 40. The proximal end 44 can also be configured with a screw head or similar structure configured to allow torque to be applied to the minimally invasive patient reference device 40. The screw head can be configured in one or more of a round design with a diametric slot, cross, hexagon, or recessed with a cross or hexagon. The proximal end 44 can also be configured with a bolt head or similar structure configured to allow torque to be applied to the minimally invasive patient reference device 40.

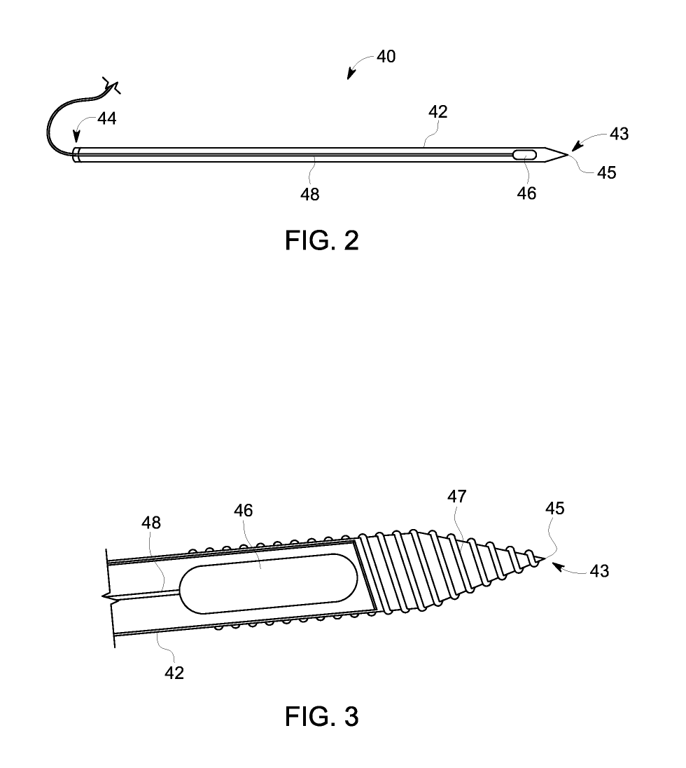

Referring now to FIG. 3, some embodiments of the distal end 43 of a minimally invasive patient reference device 40 are illustrated. FIG. 3 illustrates the distal end 43 of the elongate body 42, the microsensor 46, and the point 45. FIG. 3 also illustrates some embodiments of a placement of the microsensor 46 inside a lumen of the distal end 43 of the elongate body 42. The microsensor 46 can be disposed in the distal end of 43 of the elongate body 42 in other configurations. For example, in yet other embodiments, the microsensor 46 can be configured to be disposed on an exterior of the elongate body 42. The microsensor 46 can also be configured to be disposed in a recess in the distal end 43 of the elongate body 42.

In some embodiments, the point 45 can be configured as a sharp needle point effective for piercing the surface of the patient's skin 26 at an area proximate to the surgical field of interest 22 in a minimally invasive manner. The point 45 can also be configured as a sharpened bevel point effective for piercing the surface of the patient's skin 26 at an area proximate to the surgical field of interest 22 in a minimally invasive manner. The point 45 can be configured to pierce the surface of the patient's skin 26 and to make an incision in the patient's tissue to allow the elongate body 42 to traverse the patient's tissue to access the surgical field of interest 22. The point 45 can also be configured to allow the elongate body 42 to pass through the patient's skin and to pass through the patient's tissue to access the surgical field of interest.

In some embodiments, the point 45 can be configured as a sharp needle point effective for anchoring the minimally invasive patient reference device 40 to an anchor point on the patient's anatomy 24. The point 45 can also be configured as a sharpened bevel point effective for anchoring the minimally invasive patient reference device 40 to an anchor point on the patient's anatomy 24. In some embodiments, the point 45 can further comprise threads 47 configured to facilitate anchoring the minimally invasive patient reference device 40 to an anchor point on a patient's anatomy 24. The threads 47 can be configured as self-tapping threads, square threads, acme threads, and/or buttress threads. In some embodiments, the threads 47 can comprise a surface of the distal end 43. The threads 47 can also comprise less than or more than half of a surface of the elongate body 42.

The microsensor 46 can also be configured to be anchored internally in a bony anatomy 24. In yet other embodiments, the distal end 43 of a minimally invasive patient reference device 40 can be configured to anchor to a bony anatomy 24 such that the distal end 43 is anchored completely into the bony anatomy 24 and the microsensor 46 is partially or completely embedded into the bony anatomy 24.

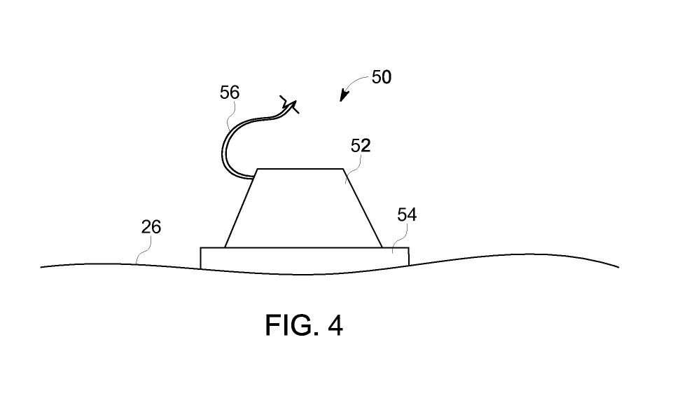

Referring now to FIG. 4, some embodiments of a transmitter 50 are illustrated. The transmitter 50 can comprise a transmitting unit 52. The transmitting unit 52 can be configured to transmit signals to the microsensor 46. The transmitting unit 52 can also be configured to transmit electromagnetic signals to the microsensor 46. The transmitting unit 52 can also be configured to transmit signals to the navigated surgical tool 60. In some embodiments, the transmitting unit 52 can be configured to transmit electromagnetic signals to the navigated surgical tool 60. In other embodiments, the transmitting unit 52 can be configured to receive signals from the navigated surgical tool 60. In yet other embodiments, the transmitting unit 52 can be configured to receive electromagnetic signals from the navigated surgical tool 60.

In some embodiments, the transmitter 50 can be configured to overcome some of the potential limitations of the microsensor 46. In general, the ability of a microsensor 46 to receive a signal or transmit a signal can diminish with a decreasing size of the microsensor 46. In particular, the ability to transmit a signal can be limited for a microsensor 46 configured to be small enough to fit in a minimally invasive patient reference device 40. Likewise, the ability to receive a signal can be limited for a microsensor 46 configured to be small enough to fit in a minimally invasive patient reference device 40. The system 10 can be configured to overcome some of the potential limitations of the microsensor 46 by configuring the microsensor 46 to receive signals and by configuring the transmitter 50 to transmit signals and to be attached proximate to the surgical field of interest 22, thereby positioning the transmitter 50 proximate to the microsensor 46. In other configurations, positioning the transmitter 50 proximate to the microsensor 46 can overcome any potential limitations in the ability of the microsensor 46 to receive signals. Also, the transmitter 50 can be configured to transmit signals of sufficient strength to penetrate the patient's skin 26 and any intervening tissue such that the signals can be received by the microsensor 46 and can provide sufficient number of sensitivity axes to calculate six degrees of freedom (three translational parameters and three rotation parameters). Furthermore, transmitter 50 can be configured to transmit signals that penetrate tissue without any attenuation.

In some embodiments, the transmitter 50 can further comprise an attachment pad 54 configured to detachably attach the transmitter 50 to the surface of the patient's skin 26 at an area proximate to the surgical field of interest 22 and proximate to the position of the distal end 43 of the minimally invasive patient reference device 40. The attachment pad 54 can comprise an adhesive coating configured to detachably attach a bottom surface of the attachment pad 54 to the surface of the patient's skin 26 at an area proximate to the surgical field of interest 22 and proximate to the position of the distal end 43 of the minimally invasive patient reference device 40. The attachment pad 54 can also comprise an adhesive coating configured to detachably attach a top surface of the attachment pad 54 to the transmitting unit 52. The adhesive can be configured to allow the attachment pad 54 and the transmitting unit 52 to remain in place during the navigation-assisted surgery procedure. In other aspects, the adhesive can be configured to allow the attachment pad 54 and the transmitting unit 52 to be detached after the navigation-assisted surgery procedure has been completed.

In some embodiments, the attachment pad 54 can comprise an opening configured such that the attachment pad 54 can be detachably attached to the surface of the patient's skin 26 at an area proximate to the surgical field of interest 22. The minimally invasive patient reference device 40 can then be anchored to patient's anatomy 24 through the opening. A top surface of the attachment pad 54 near the opening can be configured with markings to aid the surgeon in orienting and placing the attachment pad 54 over the surgical area of interest 22. The attachment pad 54 can also comprise a translucent material to aid the surgeon in placing the attachment pad 54 over the surgical area of interest 22.

In some embodiments, the transmitter 50 can further comprise a transmitter electrical lead 56 configured to electrically connect the transmitter 50 to the workstation 80. The transmitter electrical lead 56 can also electrically connect the transmitter 50 to a wireless module configured to wirelessly communicate with the workstation 80. In some configurations, the transmitter 50 can comprise a battery unit configured to power the transmitter 50 during the navigation-assisted surgery procedure. The battery unit can also be configured to supply electrical power to both the transmitter 50 and the minimally invasive patient reference device 40.

In some embodiments, the navigated surgical tool 60 can comprise any surgical instrument needed by the surgeon to carry out the surgery on the surgical field of interest 22. So the navigated surgical tool 60 can comprise one or more of a bone drill, an implant insertion device, a catheter, a peripherally inserted central catheter, a stent, and/or a guide wire. The navigated surgical tool 60 can also comprise any surgical tool commonly used in any percutaneous procedure such as angioplasty, stenting, and/or spine surgery such as a bone tap or screw inserter.

In some embodiments, the navigated surgical tool 60 can further comprise a receiver(s) 62. Thus, the navigated surgical tool 60 can be configured to receive signals from the transmitter 50. The navigated surgical tool 60 can also comprise an electromagnetic sensor and/or an electromagnetic receiver configured to receive electromagnetic signals from the transmitter 50. The navigated surgical tool 60 can be configured to provide sufficient number of sensitivity axes to calculate six degrees of freedom (three translational parameters and three rotation parameters). The navigated surgical tool 60 can also comprise magnetic pickup coils, magneto resistive sensors, fluxgate magnetometers, and/or six-degree of freedom induction microsensor(s).

In some embodiments, the navigated surgical tool 60 can comprise a tool electrical lead configured to electrically connect the navigated surgical tool 60 to the workstation 80. The tool electrical lead can also electrically connect the navigated surgical tool 60 to a wireless module configured to wirelessly communicate with workstation 80.

As shown in FIG. 1, the imaging assembly 70 can comprise any imaging system configured to image the surgical field of interest 22. The imaging assembly 70 can therefore comprise an imaging system configured to obtain a two-dimensional image, a three-dimensional image, and/or a multi-dimensional image of the surgical field of interest 22. The imaging system 70 can also comprise an imaging system configured to obtain an image in near real-time or real-time using known imaging technologies such as computed tomography (CT), magnetic resonance imaging (MRI), fluoroscopic images, positron emission tomography (PET), ultrasound, X-ray, or any other suitable imaging technology, as well as any combinations thereof. The imaging system 70 can comprise an imaging system configured to both obtain a high-resolution pre-operative multi-dimensional and to obtain near real-time or real-time images during surgery. In some aspects, imaging system 70 can comprise a C-arm, mini C-arm, or cone beam computed tomography imaging system.

As shown in FIG. 1, the workstation 80 can comprise any type of computer or processor suitable for determining a position and orientation of the patient's anatomy 24 by measuring electromagnetic signals from the transmitter received by the anchored microsensor, determining registration parameters between the global navigation reference frame and the image reference frame, and continuously correcting the registration parameters to compensate for transmitter movement by applying a real time correction factor to an initial image registration frame. The workstation 80 can also encompass many types of computer system configurations, including personal computers, hand-held devices, multi-processor systems, microprocessor-based or programmable consumer electronics, network PCs, minicomputers, mainframe computers, and the like. In some embodiments, the workstation 80 can further comprise a display 82 configured to display images of the surgical field of interest 22 and an overlay of the navigated surgical tool 60. The display 82 can comprise one or more of a computer display, a computer monitor, an LCD panel display, a CRT monitor, an OLED monitor, or other suitable display. The workstation 80 can also be configured as a portable cart with wheels.

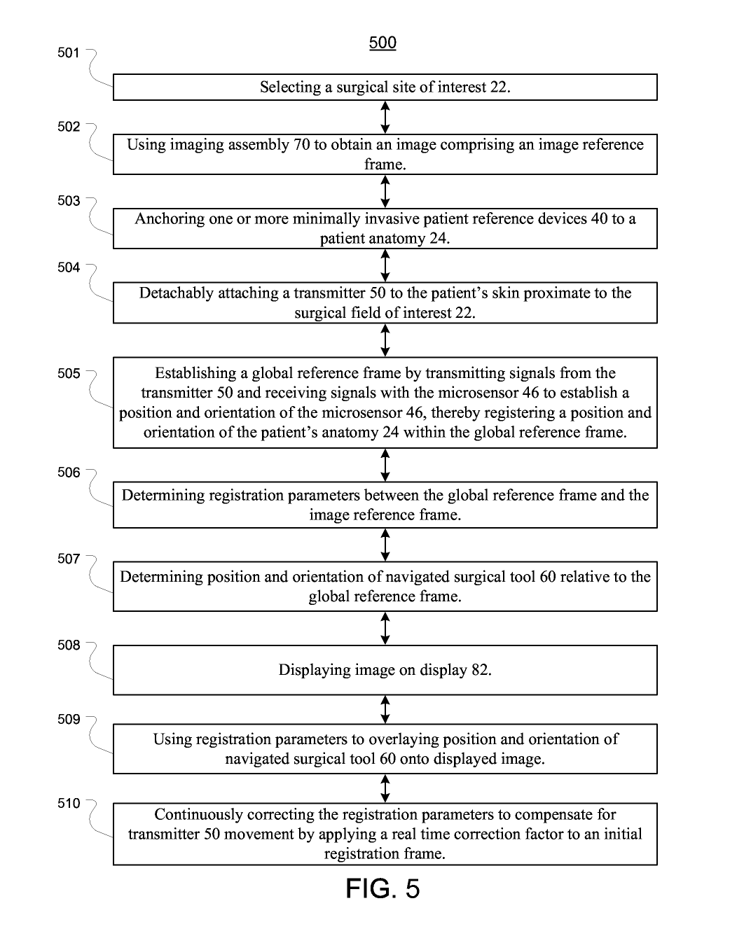

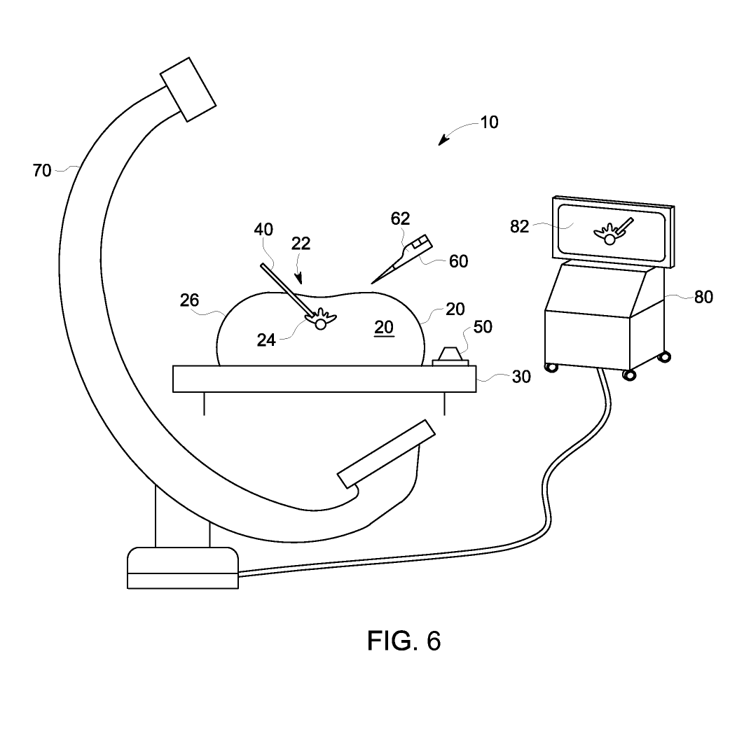

FIG. 5 contains a flow diagram illustrative of some embodiments of method 500 for navigation-assisted surgery with a minimally invasive patient reference device 40. In these embodiments, patient 20 has been prepared for surgery and is in place on table 30. As shown in box 501, the method begins by selecting a surgical field of interest 22. This process can comprise determining what type of surgery that the patient needs, considering the position of the patient 20, and considering the patient anatomy 24 into which the minimally invasive patient reference device 40 will be anchored. This process can further comprise determining what type of patient anatomy 24 will be used to anchor the minimally invasive patient reference device 40. For example, this process can include determining whether the minimally invasive patient reference device 40 will be anchored into bony anatomy or a soft tissue structure. Soft tissue structures can comprise non-mineralized tissues and can include muscle tissue, nervous tissue, and epithelial tissue.

Method 500 continues in box 502 where an imaging assembly 70 is used to obtain an image comprising an image reference frame. This process can comprise obtaining a two-dimensional, three-dimensional, or multi-dimensional image of the surgical field of interest 22. This process can further comprise obtaining an image of the surgical field of interest 22 including the patient anatomy 24. This process can also comprise obtaining a high resolution pre-operative image and continuously obtaining lower resolution images during the surgical procedure. In some embodiments, this process can comprise continuously obtaining lower resolution images during the surgical procedure. This process can comprise obtaining one or more of computed tomography (CT), magnetic resonance imaging (MRI), fluoroscopic images, positron emission tomography (PET), ultrasound, and X-ray images.

Method 500 can continue, as shown in box 503, by anchoring one or more minimally invasive patient reference devices 40 to a patient anatomy 24. This process can comprise positioning the minimally invasive patient reference device 40 proximate to the surgical field of interest 22. The point 45 can be placed against the patient's skin 26 and the point 45 can be used to pierce the patient's skin 26. The point 45 can then be inserted through the patient's skin 26 and any intervening tissue can be traversed as the minimally invasive patient reference device 40 is inserted. Once the point 45 contacts the patient's anatomy, the point 45 can be anchored to the patient's anatomy. In some aspects, the point 45 and the distal end 43 can be embedded in the patient's anatomy. The microsensor 46 can be partially or completely embedded in the patient's anatomy. Similarly, the minimally invasive patient reference device 40 can also be completely embedded in the patient's anatomy. Also, more than one minimally invasive patient reference device 40 can be anchored proximate to the surgical field of interest 22. For example, in spine surgery, a surgeon may anchor individual minimally invasive patient reference devices 40 in each of several vertebras.

In some embodiments, the patient's anatomy 24 can comprise bony anatomy such as a vertebra. For example, in spine surgery involving a vertebra, the minimally invasive patient reference device 40 can be configured as a bone screw with the microsensor 46 disposed at the distal end 43. The bone screw can pierce the patient's skin 26, traverse any intervening tissue, and point 45 can contact the spinous process of the vertebra. The bone screw can then be anchored to the spinous process by applying rotational torque and allowing the self-tapping threads to embed the distal end 43 into the spinous process, thereby embedding the microsensor 46 into the bony anatomy 24 (the spinous process). The bone screw can be embedded into other structures on the vertebra such as the transverse process.

Once the minimally invasive patient reference device 40 has been anchored, an image of the surgical field of interest 22 can be obtained to register the position and the orientation of the microsensor 46 with respect to the patient anatomy 24. An image of the surgical field of interest 22 with the anchored microsensor can be used to aid in registering the position and orientation of the microsensor 46 with respect to the image reference frame.

The method continues, as shown in box 504, by detachably attaching a transmitter 50 to the patient's skin proximate to the surgical field of interest 22. This process can comprise detachably attaching a transmitter 50 to the patient's skin proximate to the surgical field of interest 22 in a manner such that the signals transmitted by the transmitter 50 can be received by the microsensor 46. The location to which the transmitter 50 can be attached can be selected to allow the signals from the transmitter 50 to be received by the microsensor 46 and to provide sufficient number of sensitivity axes to calculate six degrees of freedom (three translational parameters and three rotation parameters).

In some embodiments, this process can comprise detachably attaching transmitter 50 to the patient's skin 26 with an attachment pad 54. This process can also comprise attaching the transmitter 50 to the patient's skin 26 with an adhesive attachment pad 54. In some aspects, a top surface of an adhesive attachment pad 54 can be affixed to transmitter 50 and a bottom surface of the adhesive attachment pad 54 can be detachably attached to the patient's skin 26.

In some configuration the adhesive attachment pad 54 can comprise a marked opening. A bottom surface of the adhesive attachment pad 54 can be detachably attached to the patient's skin 26 by using the marked opening to orient the attachment pad 54 over the surgical field of interest 22. Then the minimally invasive patient reference device 40 can be anchored through the marked opening. Once the minimally invasive patient reference device 40 is anchored through the marked opening, the transmitter 50 can be attached to a top surface of the adhesive attachment pad 54.

Method 500 continues in box 505 by establishing a global reference frame using transmitting signals from transmitter 50 and receiving signals with microsensor 46 to establish a position and orientation of microsensor 46, thereby registering a position and orientation of patient's anatomy 24 within the global reference frame. In some embodiments, this process can comprise transmitting electromagnetic signals from transmitter 50 to establish a global reference frame. By receiving these electromagnetic signals with microsensor 46, the position and the orientation of microsensor 46 relative to the global reference frame can be determined by workstation 80. Determining the position and orientation of microsensor 46 can also indicate the position and orientation of patient's anatomy 24 with respect to the global reference frame by taking into account the position and orientation of patient's anatomy 24 with respect to the position and orientation of microsensor 46. As described herein, the position and orientation of patient's anatomy 24 with respect to the position and orientation of microsensor 46 can be established by imaging patient anatomy 24 with the anchored microsensor 46.

Method 500 continues, as shown in box 506, by determining registration parameters between the global reference frame and the image reference frame. In some embodiments, workstation 80 can be used to determine the registration parameters between the global reference frame and the image reference frame. Registration parameters can comprise the translational and rotational relationships between the global reference frame and the image reference frame. Registration parameters can also comprise the three translational and the three rotational relationships between the global reference frame and the image reference frame. Registration parameters between the global reference frame and the image reference frame can also be determined by using one or more fiducial markers. In other aspects, registration parameters between the global reference frame and the image reference frame can be determined by using the patient's anatomy 24 as a fiducial marker.

Method 500 continues in box 507 by determining a position and an orientation of a navigated surgical tool 60 relative to the global reference frame. In some embodiments, this process can comprise determining a position and an orientation of a navigated surgical tool 60 in similar fashion as determining a position and an orientation of the microsensor 46 in box 505. A position and an orientation of a navigated surgical tool 60 relative to the global reference frame can be determined by transmitting electromagnetic signals from transmitter 50 and receiving these signals by a receiver 62 of the navigated surgical tool 60. By receiving these electromagnetic signals with receiver 62, the position and the orientation of navigated surgical tool 60 relative to the global reference frame can be determined by workstation 80. In some embodiments, the receiver 62 can comprise induction sensors configured to translate the relative electromagnetic signal received by the receiver 62 into a position and an orientation of the navigated surgical tool 60. Additionally, more than one navigated surgical tool 60 can be employed in the surgery with the corresponding position and orientation of each navigated surgical tool 60 determined.

Method 500 continues, as shown in box 508, by displaying the image on display 82. This process can comprise displaying a pre-operative image in real-time or near real-time image. Displaying the image can further comprise displaying a composite image of a pre-operative image and a real-time or near real-time image. Displaying the image can also include displaying the image color with false color or other contrasting coloring system. Displaying the image can comprise refreshing the image with updated images as the updated images are obtained.

Method 500 continues by using the registration parameters to overlay a position and an orientation of navigated surgical tool 60 onto an image displayed on display 82, as shown in box 509. As described above in box 506, the registration parameters between the global reference frame and the image reference frame can be determined. Likewise, box 507 describes determining a position and an orientation of a navigated surgical tool 60 relative to the global reference frame. Therefore, in some embodiments, box 509 can comprise overlaying a position and orientation of navigated surgical tool 60 onto a displayed image by providing a position and orientation of a navigated surgical tool 60 in the global reference frame and registering the position and orientation of navigated surgical tool 60 to the image reference frame. The position and orientation of navigated surgical tool 60 can be registered from the global reference frame to the image reference frame by applying the registration parameters. Applying the registration parameters can also comprise applying the registration parameters related to translational and rotational relationships between the global reference frame and the image reference frame. Once the position and orientation of navigated surgical tool 60 has been registered to the image reference frame, the position and orientation of navigated surgical tool 60 can be overlaid onto the displayed image.

In some embodiments, the displayed image can be registered to the global reference frame by applying the registration parameters. The registered image can then displayed. The position and orientation of the navigated surgical tool 60 in the global reference frame can then be overlaid onto the displayed image.

Method 500 also includes periodically or continuously correcting the registration parameters to compensate for movement of the transmitter 50 by applying a real time correction factor to an initial registration frame, as shown in box 510. In some embodiments, the transmitter 50 can experience small movements during the navigation-assisted surgery. These small movements can correspond to small movements of the patient corresponding to respiration or other cardiovascular activity such as heart pumping or blood flow, flexibility of skin, tissue removal during surgery, or displacement of tissue during surgery. The registration parameters can be continuously corrected to compensate for any small movements of the transmitter 50.

In some embodiments, the registration parameters can be continuously corrected by applying a real time correction factor to an initial registration frame. The relationships between the various components of the system 10 and corresponding reference frames can be considered in terms of mathematical transformations. The mathematical transformation of the receiver 62 to the image reference frame can be represented as T₀. The mathematical transformation of the transmitter 50 to the microsensor 46 can be represented as T₁. The mathematical transformation of the microsensor 46 to the image reference frame can be represented as T₂. The mathematical transformation of the transmitter 50 to the image reference frame can be represented as T₃. For proper determination of registration parameters, the system 10 has to ensure that the mathematical transformation of the microsensor 46 to the image reference frame remain constant (T₂=constant). To ensure that T₂ remains constant, the registration parameters can be continuously corrected in the following manner:

First, an initial image registration transformation is determined at an initial time, t₀. The initial image registration transformation is represented as T₃(t₀). The image registration transformation at a time, t, can be represented as:

T₃=T₂*T₁ [Equation 1]

Here, T1 equals the inverse of the position and orientation of microsensor 46 in the global reference frame.
Next, T₂ can be represented as:

T₂=T₃(t₀)*T₁(t₀)⁻¹ [Equation 2]

Then, Equation 2 can be substituted into Equation 1:

T₃(t)=T₃(t₀)*T₁(t₀)⁻¹*T₁(t) [Equation 3]

Lastly, Equation 3 can be used to determine the image registration transformation at time t, T₃(t), by using the initial image registration transformation, T₃(t₀), and a real time correction factor, T₁(t₀)⁻¹*T₁(t). In yet other embodiments, the Equation 3 can be used to continuously correct the registration parameters.

In some embodiments, continuously correcting the registration parameters to correct for small movements of transmitter 50 due to respiration or other cardiovascular activity such as heart pumping or blood flow, flexibility of skin, tissue removal during surgery or displacement of tissue during surgery can be used for navigation-assisted surgery where patient's anatomy 24 comprises a soft tissue structure. In some aspects, where microsensor 46 is anchored to a soft tissue structure, there can be more likelihood that transmitter 50 can experience small movements. In these cases, there can be more likelihood that transmitter 50 can experience small movements due to respiration or blood flow. Where microsensor 46 is anchored to a soft tissue structure, system 10 can be configured to continuously correct the registration parameters for periodic movement such as that due to respiration or heart pumping. System 10 can be configured to continuously correct the registration parameters to correct for periodic movement such as that due to respiration or heart pumping by incorporating a measurement of the periodic movement into the correction determination.

Referring now to the embodiments shown in FIG. 6, the transmitter 50 can be disposed proximate to the surgical field of interest 22 without being attached to patient's skin 26. Transmitter 50 can be disposed on table 30 and be disposed proximate to surgical field of interest 22. Transmitter 50 can also be configured to be disposed in a recess or other similar structure within table 30 such that transmitter 50 is proximate to the surface of the patient's skin 26 and proximate to the minimally invasive patient reference device 40. Transmitter 50 can also be disposed on a support structure such as a mechanical arm to position transmitter 50 proximate to surgical field of interest 22.

Using the systems and methods described herein eliminates the need for a relatively large external patient reference device and the resulting invasive procedure for rigidly connecting the external patient reference device to patient anatomy. They also can eliminate the need for an incision, tissue removal, and tissue damage required for placing a conventional external reference device, as well as eliminate the set up time and effort required to place a conventional external reference device and the risk of accidental impact or displacement of the conventional external reference device once placed. Additionally, the systems and methods disclosed here allow for the use of a minimally invasive patient reference device where surgical fields are not proximate to bony anatomy and where the minimally invasive patient reference device can be anchored to soft tissue structures. Further, the systems and methods disclosed here allow for continuous correction of registration parameters to correct for small movements of the transmitter due to respiration, heart pumping, or other similar movements.

The systems and methods described herein may be implemented using an existing computer processor, or by a special purpose computer processor incorporated for this or another purpose or by a hardwired system. The systems and methods may use machine-readable media for carrying out or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

The machine-executable instructions, such as program code, may be used for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the systems and methods disclosed herein. The particular sequence of such executable instructions or associated data structures represent examples of corresponding acts for implementing the functions described herein.

Embodiments of the systems and methods may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the systems and methods may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

What is claimed is:

1. A system for navigation-assisted surgery, comprising:
a patient reference device comprising an elongate body with a microsensor disposed proximate a distal end, the distal end configured to pierce a patient's skin and to be anchored in a patient's anatomy, wherein the microsensor is configured to be partially embedded in the patient's anatomy and wherein the microsensor is configured to be anchored proximate to a surgical field of interest; and
a transmitter configured to be detachably attached to the patient's skin proximate the surgical field of interest.

2. The system of claim 1, wherein the system establishes a global navigation reference frame by transmitting signals from the transmitter and receiving the signals with the microsensor to establish a position and an orientation of the microsensor, thereby registering a position and an orientation of the patient's anatomy within the global navigation reference frame.

3. The system of claim 1, wherein the patient's anatomy comprises a bony anatomy.

4. The system of claim 1, wherein the patient's anatomy comprises a soft tissue structure.

5. The system of claim 1, wherein the elongate body comprises a bone screw.

6. The system of claim 1, wherein the microsensor comprises a receiver configured to establish three translational parameters and three rotational parameters.

7. The system of claim 1, wherein the microsensor comprises one or more of an induction sensor, a magnetic pickup coil, a magnetoresistive sensor, a fluxgate magnetometer, and a six-degree of freedom induction microsensor.

8. The system of claim 1, wherein the transmitter is configured to transmit electromagnetic signals through the patient's skin to the microsensor and wherein the microsensor is configured to receive the electromagnetic signals.

9. The system of claim 1, further comprising an adhesive attachment pad configured to detachably attach the transmitter to the patient's skin.

10. A system for navigation-assisted surgery, comprising:
a patient reference device comprising an elongate body with a microsensor disposed at a distal end, the distal end configured to pierce a patient's skin and to be anchored in a patient's anatomy, wherein the microsensor is configured to be partially embedded in the patient's anatomy and wherein the microsensor is configured to be anchored proximate to a surgical field of interest;
a transmitter configured to be detachably attached to the patient's skin proximate to the surgical field of interest, the transmitter configured to establish a global navigation reference frame by transmitting signals;
an image of the surgical field of interest comprising an image reference frame;
a navigated surgical tool comprising a receiver configured to receive signals transmitted by the transmitter to track a position and an orientation of the navigated surgical tool in the global reference frame; and
a workstation configured to determine registration parameters between the global navigation reference frame and the image reference frame by determining a position and an orientation of the patient's anatomy by measuring signals from the transmitter received by the anchored microsensor and configured to convert the navigated surgical tool position and orientation from the global reference frame to the image registration frame with the registration parameters.

11. The system of claim 10, wherein the workstation is configured to correct the registration parameters to compensate for transmitter movement by applying a real time correction factor to an initial image registration transform.

12. The system of claim 11, further comprising an imaging assembly configured to obtain images to display with an overlay of the navigated surgical tool in a position and orientation registered to the image reference frame by the corrected registration parameters.

13. The system of claim 10, wherein the image comprises one or more of a CT scan, a fluoroscopic scan, an MRI scan, a PET scan, an ultrasound scan, or an X-ray scan.

14. The system of claim 10, wherein the signals are electromagnetic signals.

15. The system of claim 10, wherein the patient reference device is configured to be anchored in a patient's anatomy without removal of patient tissue.

16. The system of claim 10, wherein the patient reference device is configured to not protrude from the patient's skin once it is anchored to the patient's anatomy.

17. A system for navigation-assisted surgery, comprising:
a patient reference device comprising an elongate body with a microsensor disposed at a distal end, the distal end configured to pierce a patient's skin and to be anchored in a patient's anatomy, wherein the microsensor is configured to be partially embedded in the patient's bony anatomy and wherein the microsensor is configured to be anchored proximate to a surgical field of interest;
a transmitter configured to be detachably attached to the patient's skin proximate to the surgical field of interest, the transmitter configured to establish a global navigation reference frame by transmitting electromagnetic signals capable of being received by the microsensor;
an imaging assembly configured to obtain images of the surgical field of interest, the images comprising an image reference frame;
a navigated surgical tool comprising a receiver configured to receive the electromagnetic signals transmitted by the transmitter;
a workstation configured to determine registration parameters between the global navigation reference frame and the image reference frame by determining a position and an orientation of the patient's bony anatomy by measuring electromagnetic signals from the transmitter received by the anchored microsensor, wherein the workstation is configured to track the navigated surgical tool in the global reference frame by measuring electromagnetic signals and wherein the workstation is configured to register the navigated surgical tool position and orientation from the global reference frame to the image registration frame; and
a display configured to display the images and to display overlays of the navigated surgical tool in a position and orientation registered to the image reference frame.

18. The system of claim 17, wherein the workstation is configured to correct the registration parameters to compensate for transmitter movement by applying a real time correction factor to an initial image registration transform.

19. The system of claim 17, wherein the patient's bony anatomy comprises a spinous process of a vertebra.

20. The system of claim 17, further comprising an adhesive attachment pad configured to detachably attach the transmitter to the patient's skin.
